(19) United States
(12) Patent Application Publication
(10) Pub. No.: US 2003/0139673 A1
(43) Pub. Date: Jul. 24, 2003

(54) Illumination system

(76) Inventors: Robert L. Vivenzio, Auburn, NY (US); Raymond A. Lia, Auburn, NY (US); Allan I. Krauter, Skaneateles, NY (US); Dominick Danna, Syracuse, NY (US); Scott S. Stearns, Marietta, NY (US); Richard A. Tamburrino, Auburn, NY (US); John W. Sims, Weedsport, NY (US); Raymond P. Dromms, Liverpool, NY (US)

Correspondence Address: WALL MARJAMA & BILINSKI, 101 SOUTH SALINA STREET, SUITE 400, SYRACUSE, NY, 13202, US

(21) Appl. No.: 10/051,811

(22) Filed: Jan. 18, 2002

(51) Int. Cl. A61B 5/02

(52) U.S. Cl. 600/490

(57) ABSTRACT

An illumination system for a medical diagnostic instrument includes at least one light directing element disposed relative to an instrument housing and at least one illuminating device, such as a white LED which is optically coupled to a peripheral portion of the light directing element for providing directed illumination of a target of interest.

### FIELD OF THE INVENTION

This invention relates generally to the field of medicine, and more specifically to a compact lightweight illumination system for a medical diagnostic instrument.

### BACKGROUND OF THE INVENTION

Certain measuring apparatus are commonly known, such as sphygmomanometers, which are used in the medical field for measuring the arterial blood pressure of a patient. A typically known blood pressure measuring device includes an inflatable sleeve which is wrapped around a limb (e.g., an arm or leg) of a patient. The device includes a gage that is pneumatically interconnected to the inflatable sleeve. A bellows or diaphragm assembly is sealingly contained within the interior of the gage along with a movement mechanism having an indicating member which is disposed in relation to a dial face having suitable measuring indicia. In brief, pressure variations which occur as the sleeve is inflated and deflated by a pneumatic bulb or other inflation apparatus causes movement in the bellows assembly which is then converted by the movement mechanism into a corresponding circumferential movement of the indicating member. The user can then read the indicating member relative to the dial face in combination with listening to the heart sounds via a stethoscope in order to obtain a blood pressure measurement.

It is often required to take a number of patient blood pressure readings during the course of a typical hospital visit, such as before and after surgery or other medical procedure. The need to perform these measurements is often an inconvenience for the patient. This situation is exacerbated at night, given that the caretaker will awaken the patient and others in a darkened hospital room by turning the room lights on before even beginning a measurement procedure. That is to say, all presently known pressure measuring devices of the above type require ambient light in order to adequately read the relative position of the indicating member relative to the dial face.

Other medical diagnostic apparatus, including handheld instruments such as otoscopes and ophthalmoscopes, typically include an incandescent halogen bulb or other suitable light source which is powered by batteries contained within the handle of the instrument or include a remote light source such as a light box which provides illumination through a series of transmitting optical fibers. While the use of a conventional bulb, either with or without the further use of associated fiber-optics, is adequate to generate proper illumination of a medical target such as the ears, eyes, throat or other body cavity of a patient, there is a compelling need in the field to employ other illumination sources, such as white light emitting diodes (LEDs), which require less power, produce less heat, and are more inexpensive to incorporate. A general problem with such light sources, however, is that the output illumination of these devices is limited and therefore it is required to properly conduct or direct light to a to-be observed target.

Miniature light sources such as LEDs and laser diodes are known illumination sources that are often preferred because they have low power consumption and longer life than halogen bulbs. However, it is difficult, for example in a medical device, to properly conduct light from such sources effectively relative to a target of interest.

### SUMMARY OF THE INVENTION

It is therefore a primary object of the present invention to overcome the above-noted problems of the prior art.

It is another primary object of the present invention to provide a medical measuring apparatus which permits a physician, care giver, or other user to perform a measurement or diagnostic procedure without requiring ambient illumination.

It is yet another primary object of the present invention to provide a diagnostic apparatus which permits suitable illumination using at least one miniature illumination device, such as an LED, to be better directed to a medical target area, such as the ears, eyes, throat, or other body cavity without the need for optical fibers.

Therefore, and according to a preferred aspect of the present invention, there is provided an illumination system for a medical device, said system comprising:

a light directing element disposed in relation to an instrument housing; and

at least one illumination device coupled to a peripheral portion of said light directing element for providing illumination through said element to enable a user to view a target of interest.

Preferably and according to one embodiment, the medical device is a sphygmomanometer in which a transparent viewing window covering the dial face of a gage can be used as a light directing element. At least one LED, preferably a white LED, is optically coupled to an edge of the viewing window, causing the illumination output to be transmitted peripherally along an edge thereof. More particularly, the at least one LED can be inset within a receiving portion of the gage so as to directly impinge on the periphery of the viewing window. Preferably, the edge of the viewing window is coated with a non-reflective material to more efficiently and uniformly transmit illumination.

The light directing element can assume other forms whereby light can be directed from an optically coupled LED or other miniature illumination device, such as a laser diode, to provide output relative to a target of interest. For example, at least one LED can be optically coupled to a conical speculum to provide light directly to the outer ear in an otoscope, or similarly can be directed to the peripheral edges of an intra-oral dental mirror or other similar device to permit improved visibility of the mouth or throat.

According to a preferred version, at least one lens or lenslet can be provided in relation to the LED, such as, for example, integrally on the proximal end of a speculum for collimating or converging light emitted from an adjacently or proximately disposed LED. In addition to this version, a low refractive index coating can be applied to the speculum in order to effectively convert the speculum or other light directing element into a light guide.

According to another preferred aspect of the present invention, there is provided a medical diagnostic instrument comprising a housing, a light directing element disposed in relation to said housing, and at least one illumination device optically coupled to a peripheral portion of said at least one light directing element for providing illumination at a target of interest.

According to yet another preferred aspect of the present invention, there is provided a pressure measuring device comprising: a housing, a pressure responsive member disposed within said housing, said pressure responsive member having a movable surface, an indicating member which is caused to move based upon a change in said movable surface of said pressure responsive member, a dial face having indicia against which said indicating member moves, and an illumination system including at least one illumination device and at least one light directing element optically coupled to said at least one illumination device for providing illumination to enable a user to view the dial face and the indicating member during a measurement without requiring ambient light.

According to still another preferred aspect of the invention, there is provided a method for illuminating a medical target, said method comprising the steps of: coupling at least one LED to a peripheral portion of a light directing element of a medical diagnostic instrument; and illuminating said at least one illumination device, such as an LED, so as to uniformly direct the light from said at least one LED towards said medical target.

One advantageous feature of the present illumination system is that blood pressure readings can be reliably performed in, for example, a darkened hospital room, using either a wall or sleeve mounted apparatus without first requiring a physician or other care giver to first have to turn the room lights on. This minimizes considerable inconvenience to the patient.

Another advantageous feature of the present invention is that the LED(s) or other illumination device can be activated automatically without first having to manually activate a switch or other operating member. Furthermore, the device can include a timing circuit such that illumination can also be discontinued automatically after a predetermined time interval.

The herein described illumination system is advantageous in that the LEDs and batteries are capable of a longer useful life than halogen bulbs and/or optical fibers used in previously known systems.

Another advantage is that utilizing lenses in relation to an LED or other illumination device permits enhanced optical coupling so as to direct the light along a preferred or optimal path to better illuminate a target of interest.

Yet another advantage is that the proposed illumination system is more durable and shock resistant than previously known systems.

These and other objects, features, and advantages will be readily apparent from the following Detailed Description which should be read in conjunction with the accompanying drawings.

### BRIEF DESCRIPTION OF THE DRAWINGS

FIG. 1 is a top perspective view of a blood pressure measuring apparatus made in accordance with a preferred embodiment of the present invention;

FIG. 2 is a partial sectional view, taken in elevation, of the pressure measuring apparatus depicted in FIG. 1;

FIG. 3 is a partial top perspective exploded view of the gage of the pressure measuring apparatus of FIGS. 1-2;

FIG. 4 is a partial bottom view of the peripheral bumper of the gage depicted in the apparatus of FIG. 3;

FIG. 5 is a front view of a wall-mounted blood pressure measuring apparatus in accordance with a second embodiment of the present invention;

FIG. 6 is a partial exploded top perspective view of a portion of the blood measuring device of FIG. 5;

FIG. 7 is a top view of a blood pressure measuring apparatus in accordance with a third embodiment of the invention;

FIG. 8 is a partially exploded perspective view of an otoscope including an illumination system made in accordance with an embodiment of the invention;

FIG. 9 is a rear perspective view of the speculum portion of the otoscope of FIG. 8;

FIG. 10 is a partially cutaway rear perspective view of the otoscope of FIGS. 8 and 9;

FIG. 11 is a partial sectioned view of a speculum portion made in accordance with a preferred embodiment for the otoscope of FIG. 8;

FIG. 12 is a partial sectioned view of a speculum portion made in accordance with another preferred embodiment for the otoscope of FIG. 8;

FIG. 13 is a rear perspective view, partly in section, of a speculum portion in accordance with another embodiment of the invention;

FIG. 14 is a partial perspective view of an intra-oral dental mirror having an illumination system made in accordance with the present invention; and

FIG. 15 is an enlarged view, partly in section, of the handle of the intra-oral dental mirror of FIG. 14; and

FIGS. 16 and 17 are partial side elevation views of embodiments related to the coupling of an LED with a dental mirror.

### DETAILED DESCRIPTION

The present invention has been particularly shown and described with reference to certain measuring apparatus, and particularly to medical diagnostic devices such as otoscopes for diagnosing the outer ear, sphygmomanometers for measuring the blood pressure of a patient, and dental mirrors used in oral examinations. It will be understood, however, by one skilled in the art that other apparatus, including non-medical devices used for measuring pressure, force, and other similar parameters can easily incorporate the inventive concepts recited herein.

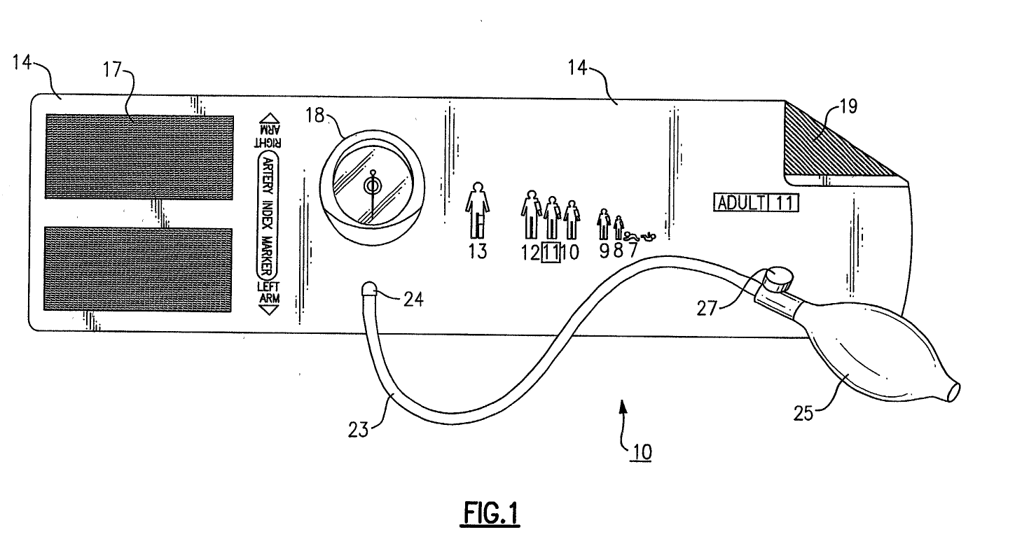

According to a first embodiment and referring herein to FIGS. 1 and 2, there is shown a blood pressure measuring apparatus 10 including an inflatable sleeve or cuff 14 which is sized to be wrapped about the limb of a patient (not shown) in a manner which is commonly known such as by using hook and loop fasteners 17, 19 disposed on opposing adjacent sides of the sleeve. The sleeve 14 can, for example, be appropriately sized to fit about the limb (e.g., arm or leg) of adults, children, or neonatal patients and is preferably made from a fluid impermeable material such as polyethylene, polyamide and the like. A preferred inflatable sleeve useful in the present embodiment is a bladderless sleeve made from a pair of RF welded sleeve portions as described in U.S. Pat. No. 6,036,718, the entire contents of which are herein incorporated by reference. It should be understood that the nature of the inflatable sleeve 14 itself, however, is not critical to an understanding of the present invention, and in fact either bladderless or sleeves having bladders can be used.

The inflatable sleeve 14 of the present embodiment is made from a pair of sleeve portions 32, defining an enclosed interior 30 which provides an expandable pressure chamber fluidly connected by means of a receiving port 24 to a depressable pneumatic bulb 25 serving as an inflation/deflation apparatus through a hose 23.

Referring to FIG. 2, the herein described sleeve 14 further includes a socket 22 which is formed in one of the sleeve portions 32. Preferably, the socket 22 is secured, such as by RF welding, within a slot that is formed in the sleeve portion 32. The socket 22 includes a cup-like enclosure which includes an open end and a bottom surface having a port opening 26 which permits fluid communication between a gage 18 placed within the socket and the interior 30 of the inflatable sleeve 14.

The gage 18 according to this embodiment is a substantially cylindrically shaped member having an upper portion 34 and a narrowed lower portion 38 defining a fluid-tight hollow interior. The narrowed lower portion 38 includes an engagement end 42 which is sized for sealing engagement directly with the socket 22 of the inflatable sleeve 14 and further includes an opening 54 which permits fluid from the interior 30 of the sleeve to enter the interior of the gage 18. The above attachment is extremely useful given that known gage are typically attached through tubing to the sleeve similar to the manner in which the pneumatic apparatus shown in FIG. 1 is attached thereto. The gage 18 is preferably rotatable within the socket 22, thereby permitting both left/right armed attachment of the sleeve 14, as well as, ease in use by either a care giver or patient.

Still referring to FIG. 2, the gage 18 itself retains a number of components including a pressure responsive element, such as a diaphragm 46 having a movable surface 50 which is positioned within the narrowed lower portion 38 of the gage interior along a fluid path extending to the opening 54 provided in the engagement end 42. As noted, the interior of the gage 18 is sealed so as to define a pressure chamber therein.

In short, the opening 54 in the engagement end 42 of the gage 18 permits fluid (air) to enter the housing from the interior 30 of the inflatable sleeve 14 and effect changes to the movable surface 50 of the diaphragm 46. A movement mechanism provided mainly in the upper portion 34 of the gage 18 includes an axial displaceable shaft member 68 having a lower or input end which is in substantial proximity to the movable surface 50 of the diaphragm 46.

More specifically, and referring to FIG. 2, the movement mechanism according to this described embodiment further includes a helically wound spring member 72 which is coaxially positioned over an intermediate portion of the shaft member 68. According to the present embodiment, the shaft member 68 is disposed along a vertically extending primary axis, an upper or output end 76 of the shaft member being attached to an indicating member 80 which extends horizontally relative to a dial face 84 secured by conventional means within the upper portion 34 of the gage 18. An upper end of the spring member 72 is attached to an intermediate portion of the shaft member 68 while a lower end of the spring member is attached to a fixed support. Additional details relating to the features and functioning of the movement mechanism and blood pressure measuring device are described in U.S. Pat. Nos. 5,966,829 and 6,120,458 as well as copending U.S. Ser. No. 09/929,856, the entire contents of each being herein incorporated by reference. In passing, it should be noted that the form of device, including the movement mechanism, is exemplary meaning that other suitable devices can easily be substituted in practicing the invention.

In operation, and following inflation of the sleeve 14 to a predetermined pressure level, the sleeve 14 is deflated using a bleed valve 27, provided adjacent the pneumatic bulb 25. Changes in the fluid pressure of the inflatable sleeve 14 are transmitted through the respective openings 26, 54 in the socket 22 and the engagement end 42 of the gage 18 to the movable surface 50 of the diaphragm 46, causing movement thereof as the sleeve 14 is inflated or deflated. As the movable surface 50 moves upwardly with inflation, the lower end of the shaft member 68 is caused to translate axially against the biasing of the spring member 72. As a result, the spring member 72 is caused to flex and against the restraint of the fixed support, the shaft member 68 is further caused to rotate about its primary axis. This rotation causes the upper end 76 of the shaft member 68 and the attached indicating member 80 to move circumferentially relative to measurement indicia which are provided on the dial face 84. Using a stethoscope (not shown), a blood pressure reading can be made by observing the position of the indicating member 80 relative to the measurement indicia of the dial face 84 as the pressure in the sleeve 14 is decreased.

A transparent viewing window 92 sealingly covers the top of the upper portion 34 of the gage 18 and permits viewing of the indicia provided on the dial face 84 relative to the indicating member 80.

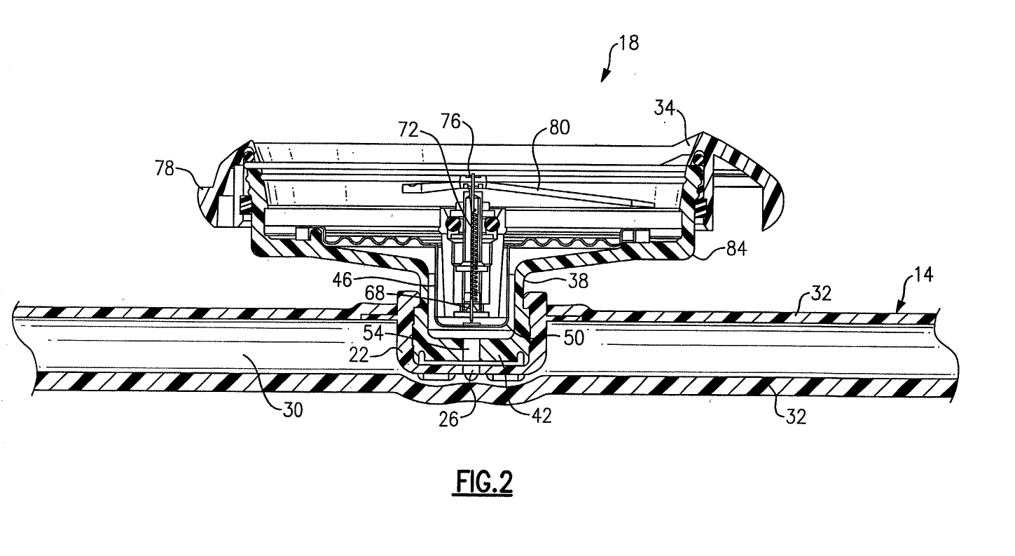

Referring to FIGS. 2 and 4, a peripheral bumper 78 is releasably attached over the exterior of the upper portion 34 of the gage housing 18, including the transparent viewing window 92, which is exposed by a center opening 79, FIG. 3. This bumper 78, which is described in greater detail in previously incorporated U.S. Ser. No. 09/929,856, assists in preventing shock or impact loads from being transmitted to the components retained within the interior of the gage housing, such as those which may occur when the housing is dropped during use. The peripheral bumper 78 includes an edge portion 81, FIG. 3, which extends above the mounting plane of the transparent viewing window 92. The transparent viewing window 92, preferably made from a moldable plastic, sealingly covers the upper portion of the above-described gage 18 and includes an outer peripheral edge 96, FIG. 3.

Referring now to FIGS. 3 and 4, a miniature white light emitting diode (LED) 100, such as a phosphor-type LED manufactured by Nichia America as described in U.S. Pat. No. 5,998,925 is disposed in substantially direct contact with a radial portion of the peripheral edge 96 of the transparent viewing window 92. For purposes of this description, a white LED is shown. It will be readily apparent that other miniature illumination devices, such as laser diodes, can also be utilized. More preferably, a notched receiving portion 106 of the gage housing 18 is removed, the portion being sized to receive the LED 100 therein. The peripheral edge 96 is also preferably frosted or otherwise coated with a non-light reflective material, such as a white coating or film though other suitable materials can be used, to more efficiently and uniformly transmit the light evenly over the entirety of the periphery of the edge 96.

Referring to FIG. 4, the peripheral bumper 78 includes an interior bottom cavity 86 which is sized to support the LED 100 and is electrically connected through wires 82 and a voltage amplifier 90 to a retained miniature battery power supply 88.

A manual switch 110 provided on the exterior of a top facing portion 114 of the peripheral bumper 78 is interconnected through a mount provided within the interior bottom cavity 86 to appropriate wiring 82 to the battery power supply 88 and the LED 100 to permit activation thereof when a blood pressure measurement procedure is being performed.

A timing circuit 130, which includes a capacitor, (not shown) is also provided within the interior bottom cavity 86 of the peripheral bumper 78 which automatically deactivates the white LED 100 after a predetermined time interval following activation using the manual switch 110. For example, thirty (30) seconds or other reasonable time period covering that of a typical blood pressure measurement time frame can be used.

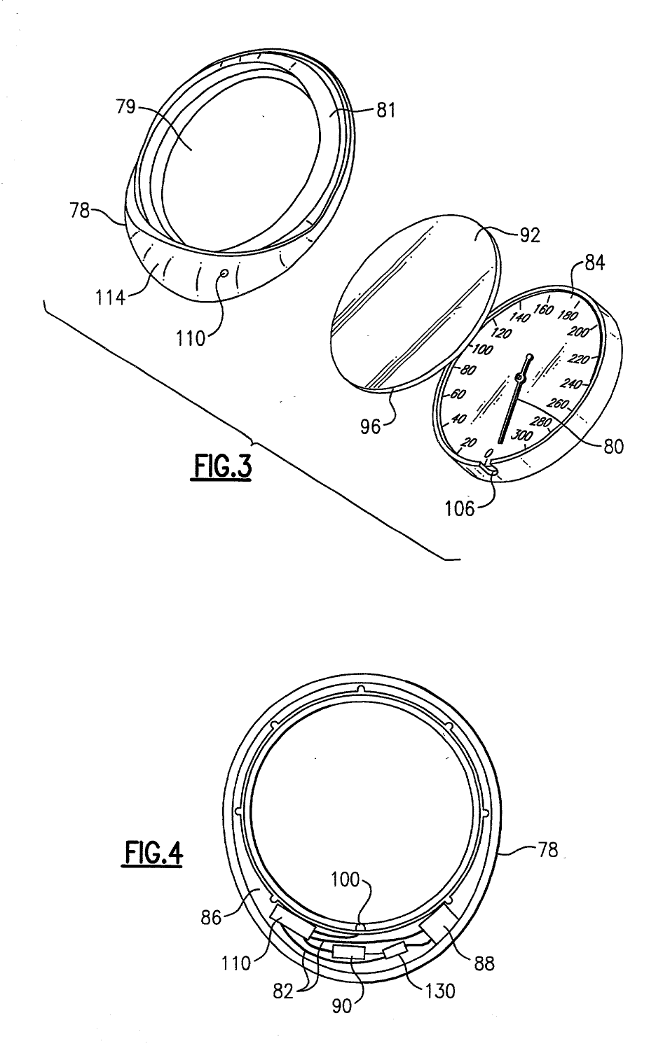

Referring to FIGS. 5 and 6, a pressure measuring apparatus 136 made in accordance with a second embodiment of the invention includes a wall or stand mounted gage 140 which is similarly interconnected to an inflatable sleeve 14, FIG. 1, using a pneumatic bulb 146 or similar inflation apparatus as tethered through a hose 149. The gage 140 of this embodiment is significantly larger than the one previously described, but the interior thereof includes similar components whereby entering fluid from the interior of the sleeve 14 causes circumferential movement of an indicating member 153 relative to measuring indicia of a dial face 159. Like the preceding, a transparent viewing window 148 includes a peripheral edge 152 which can be similarly treated with a non-reflective coating, a portion of which can be placed in proximity with at least one white LED 156.

The wall or stand mounted version according to this particular embodiment includes a different movement mechanism, shown pictorially as 160, than used in the preceding blood pressure sleeve mounted version.

According to this embodiment, the gage 140 includes an internal pressure actuable switch 120 which is opened when a predetermined pressure level is reached in order to automatically energize the white LED 156. That is, depression of the pneumatic bulb 146, will cause the white LED 156 to be activated automatically once a predetermined pressure level is achieved during inflation of the sleeve 14, FIG. 1. As in the preceding, the apparatus can further include a timing circuit 180 which de-energizes the white LED 156 after a predetermined time period, such that a manual switch is not required.

The transparent viewing window 148 can otherwise be treated to conduct light in a specific manner. As in the preceding, frosting or coating of the outer peripheral edge 152 of the window 148 with a non-reflective coating will promote scattering and uniform transmission of light. Alternately, other portions of the transparent viewing window 148 can be etched or coated so as to define a non-reflective portion in order to similarly conduct light to other portions of the window as needed. A literally infinite number of combinations are possible, such as, for example, defining an inner or central ring as well as an outer ring in which light can be uniformly transmitted.

In operation, the bulb 146 inflates the sleeve and a bleed valve 151 is used to deflate the sleeve 14, FIG. 1. Fluid changes within the sleeve interior are sensed by the movement mechanism 160 which are translated to an indicating member 154 which moves relative to indicia 157 on a dial face 159 during inflation and deflation thereof.

The pressure actuable switch 120 is fluidly connected to an input port of the gage 140 and causes activation of the white LED 156 when a predetermined pressure level is achieved during inflation. Once the LED 156 is activated, the timing circuit 180 permits the LED to maintain power for a predetermined time interval.

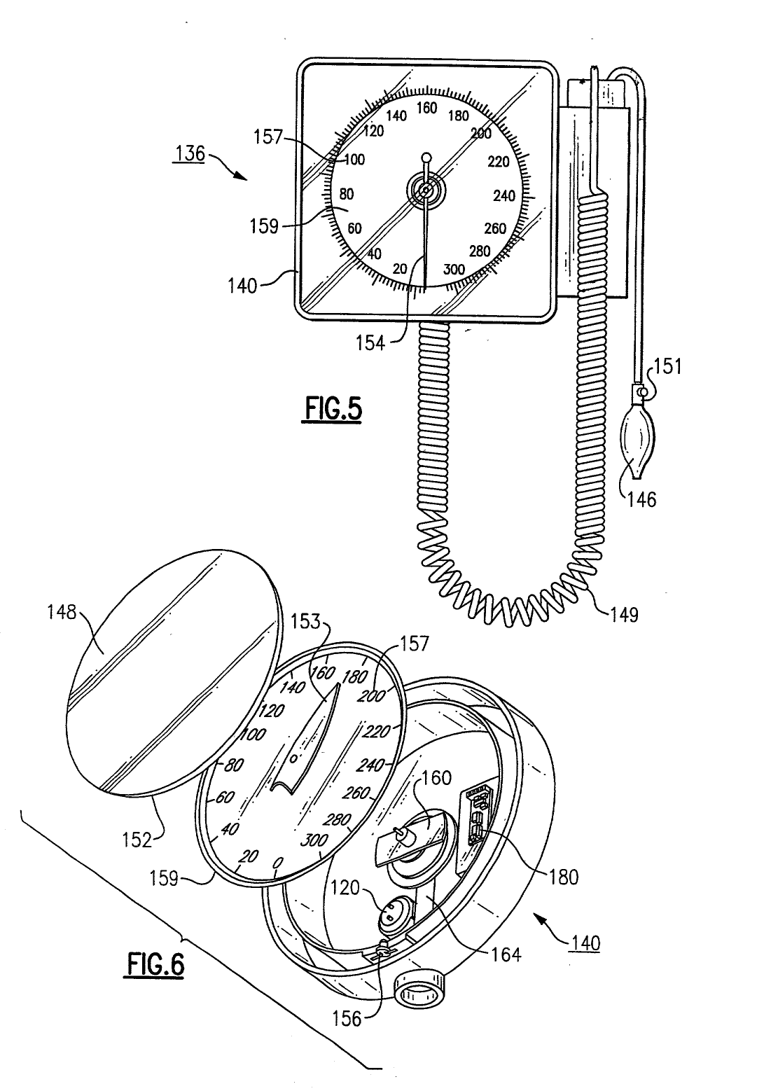

It should be apparent that other pressure or other forms of measuring devices which utilize an indicator member, regardless of the movement mechanism utilized can include an illumination system as described herein. As shown in FIG. 7, another a blood pressure measuring device 184 includes an elastomeric sleeve 186 which encloses a pneumatic bulb and a gage 190 in a unitary structure. A switch 192 is included on the exterior of the sleeve 186 which activates a white LED or LED array (not shown) that can be placed in substantial contact with the periphery of a transparent viewing window 188 in a manner similar to that previously described.

The preceding embodiments referred specifically to sphygmomanometers. Other medical diagnostic instruments, however, can include the illumination system of the present invention.

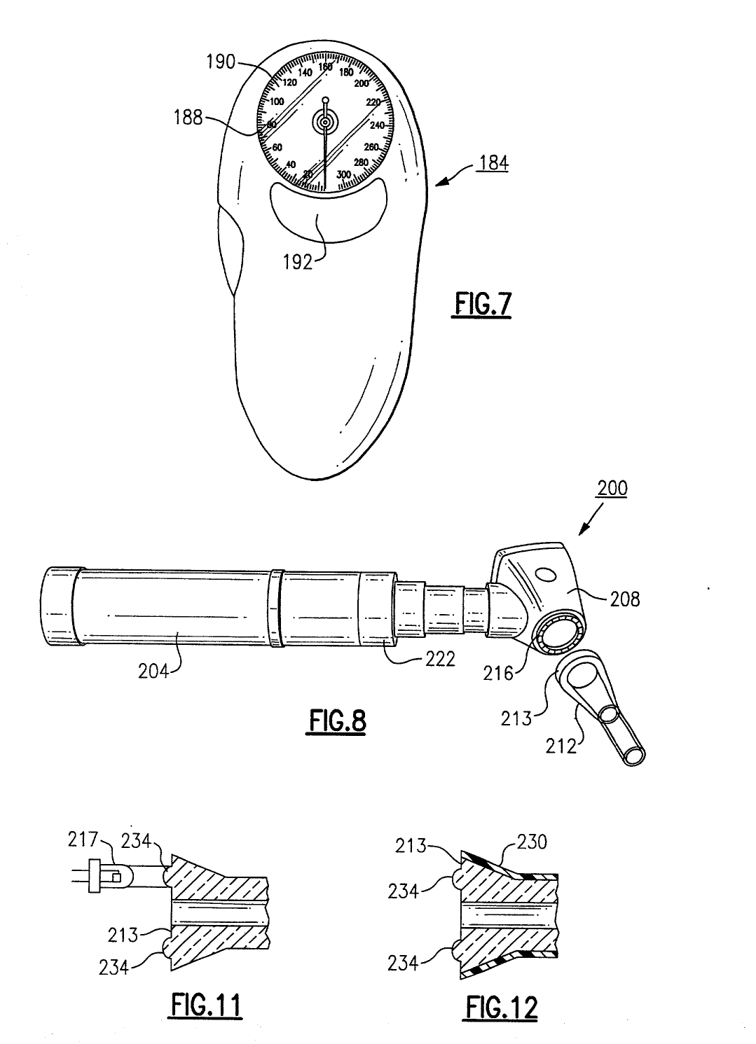

For example and referring now to FIGS. 8-13, an otoscope 200 used for examination of the outer ear of a patient can be configured using the above concepts to include an illumination system in accordance with the invention.

Referring more specifically to FIGS. 8-10, the otoscope 200 is a hand-held device which includes a cylindrical handle 204 having an instrument head 208 attached to the top of the handle. A pair of batteries (not shown) are contained within the interior of the handle 204 though electrical power could also be supplied through connection to a wall transformer (not shown) or other suitable means.

A conically shaped speculum 212, made from a transparent light directing material, such as polystyrene, is releasably attached to the distal end of the instrument head 208. A white LED array 216, including, for example, a plurality of phosphor-type LEDs manufactured by Nichia America is coupled to a proximal surface 213 of the conical speculum 212, the LED array 216 being electrically connected to the batteries of the handle 204 through conventional means.

The white LED array 216 is connected to the batteries in the handle 204 through conventional means such that the illumination LED can be adjusted through a control 222 provided in the top of the handle 204 of the instrument 200. The design and operation of the control 222 is commonly known and does not require additional description.

In operation, the speculum 212 is coupled to the distal end of the instrument head 208 and the white LED array 216 is activated when the instrument 200 is ready and the speculum 212 is inserted into the ear of a patient (not shown). The light output of the white LED array 216 is directed or channeled through the conical peripheral area of the speculum 212 to the distal end 219 and directly to the outer ear.

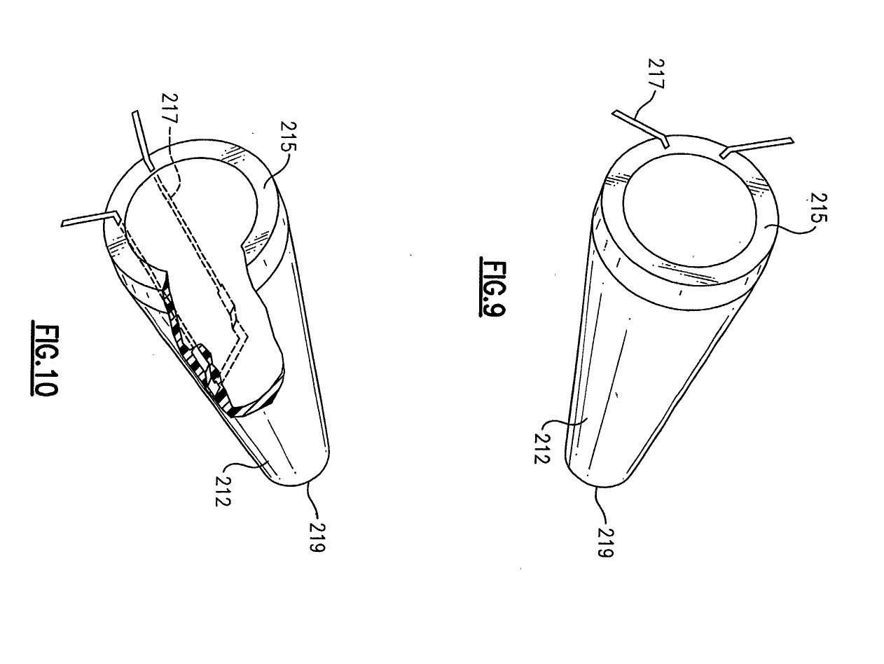

Referring to FIGS. 9 and 10, at least one white LED can be positioned anywhere along the periphery and moreover anywhere along the axial length of the speculum 212. For example, a white LED 217 is preferably imbedded into the plastic material of the speculum 212 with the proximal end of the speculum having a reflective surface 215, such as that provided by a metal foil This reflective surface increases the light that exits the speculum 212 at the distal tip 219.

Referring to FIGS. 11-13, the proximal surface 213 of the speculum 212 can include at least one integral or attached collimating or converging lens or lenslet which can collect output light from a corresponding one of the LED array 216 and direct the light to the distal tip 219, FIG. 8. In addition, and according to FIG. 12, a cladding layer 230 of a lower index of refraction material, such as acrylic, can be added to the exterior of the conical speculum 212 which in effect allows the speculum to behave similarly to an optical fiber. The layer 230 also acts as a protective layer for the speculum.

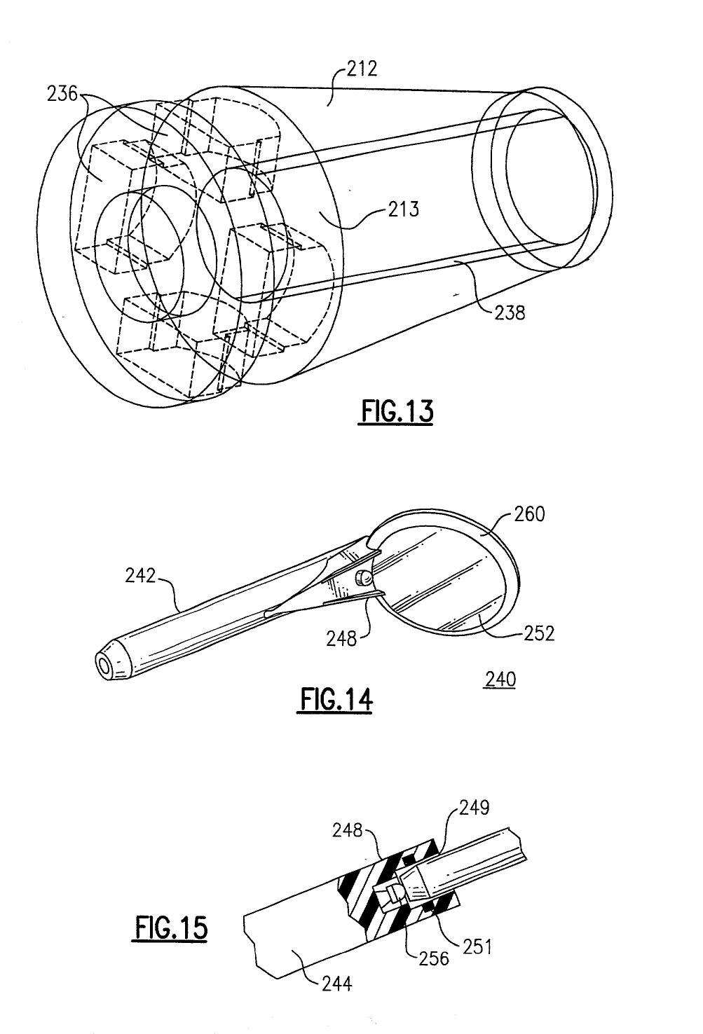

FIG. 13 illustrates a speculum which includes plurality of lens elements 236 attached to the proximal surface 213 thereof and further includes an internal sleeve 238 made from a material, such as black anodized aluminum, to mask incoming light from the inner diameter of the speculum.

It should be apparent that the type of otoscopic instrument is not restricted, that is, the herein described otoscope can be an optical version or a videoized type which includes an electronic imager either within the instrument head or within a proximal attachment.

Referring to FIGS. 14-18, an illumination system made in accordance with a further embodiment of the present invention is utilized in an intra-oral dental mirror 240.

The dental mirror 240 includes an elongated stem 242 having a distal end 248 onto which a mirrored portion 252 is fabricated. The stem 242 is fabricated from a transparent plastic material, such as styrene, so that it functions as a light guide. The mirrored portion 252 is retainingly attached to the handle 244 using an O-ring 251 and is sized to be positioned within the mouth of a patient (not shown). The mirrored portion 252 is angled relative to the axis of the handle 244 at the distal end of the stem 242. Preferably, the mirrored portion 252 is retro-flexed approximately 30° to permit viewing.

According to the invention, a miniature white LED 256 is also provided in the distal end 248 of the handle 244 and is positioned relative to the slot 249 such that the LED is coupled to the stem 242 of the mirrored portion 252. The LED 256 is powered, either through batteries provided in the handle 244 or through another suitable power source such as a wall transformer (not shown). The coupling of the LED 256 to the stem 242 of the mirrored portion 252 permits uniform illumination of the stem and of the mirrored portion 252.

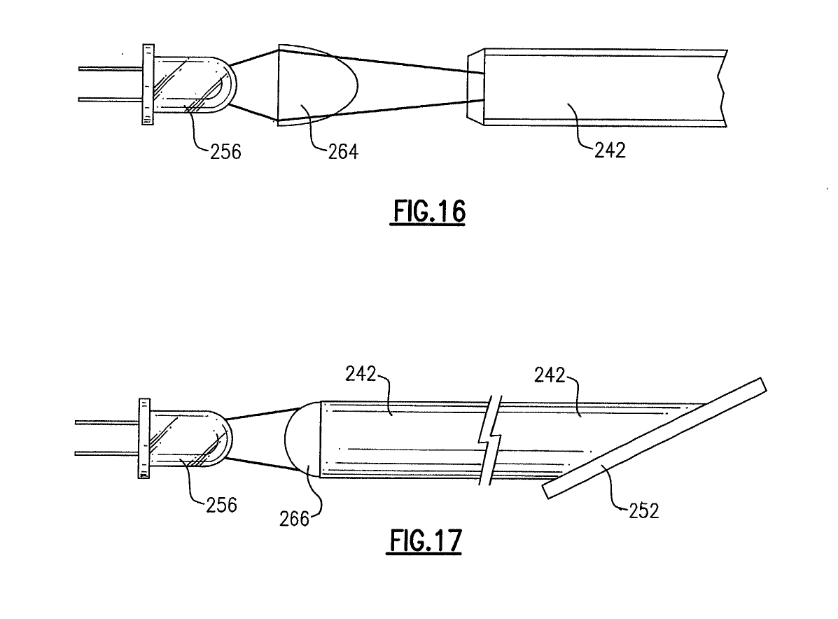

Referring to FIGS. 16 and 17, at least one aspheric collimating or converging lens element(s) 264 or 266 can be attached to or integrally provided at the proximal end of the stem 242 in the illumination path of the LED 256. This lens element 264 or 266 can be used to conduct light to the mirrored portion 252 via the stem 242. Other lens elements (not shown) in addition to those described herein can be added.

In use, the mirrored portion 252 is inserted into the mouth of a patient with the peripheral edge 260 being uniformly illuminated by the white LED 256 via the stem 242. Though only one LED is described for use in this embodiment, it should be readily apparent that additional LEDs could be utilized and coupled to the mirrored portion of the mirror in a similar manner.

### PARTS LIST FOR FIGS. 1-17

| | |
|---|---|
| 10 | blood pressure measuring apparatus |
| 14 | inflatable sleeve |
| 17 | hook and loop fastener |
| 18 | gage |
| 19 | hook and loop fastener |
| 22 | socket |
| 23 | hose |
| 24 | port |
| 25 | bulb |
| 26 | opening |
| 27 | bleed valve |
| 30 | interior |
| 32 | sleeve portions |
| 34 | upper portion |
| 38 | lower portion |
| 42 | engagement end |
| 46 | diaphragm |
| 50 | movable surface |
| 54 | opening |
| 68 | shaft member |
| 72 | spring member |
| 76 | upper end |
| 78 | peripheral bumper |
| 79 | center opening |
| 80 | indicating member |
| 81 | edge portion |
| 82 | wiring |
| 84 | dial face |
| 86 | interior bottom cavity |
| 88 | battery power supply |
| 90 | voltage amplifier |
| 92 | viewing window |
| 96 | outer peripheral edge |
| 100 | white LED |
| 106 | notched receiving portion |
| 110 | switch, manual |
| 114 | top facing portion |
| 120 | pressure actuable switch |
| 130 | timing circuit |
| 136 | pressure measuring apparatus |
| 140 | gage |
| 146 | bulb |
| 148 | viewing window |
| 149 | hose |
| 151 | bleed valve |
| 152 | peripheral edge |
| 153 | indicating member |
| 154 | indicating member |
| 156 | white LED |
| 157 | indicia |
| 159 | dial face |
| 160 | movement mechanism |
| 164 | input port |
| 180 | timing circuit |
| 184 | device |
| 186 | elastomeric sleeve |
| 188 | transparent viewing window |
| 190 | gage |
| 192 | switch |
| 200 | otoscope |
| 204 | handle |
| 208 | instrument head |
| 212 | speculum |
| 213 | proximal surface |
| 215 | reflective surface |
| 216 | white LED array |
| 217 | white LED |
| 219 | distal end |
| 222 | control |
| 226 | lenses |
| 230 | cladding layer |
| 234 | lens element |
| 236 | lens elements |
| 238 | sleeve |
| 240 | dental mirror |
| 242 | stem |
| 244 | handle |
| 248 | distal end |
| 249 | slot |
| 251 | O-ring |
| 252 | mirrored portion |
| 256 | white LED |
| 260 | peripheral edge |
| 264 | lens element |
| 266 | lens element |

We claim:

1. An illumination system for a medical diagnostic instrument, said system comprising:
at least one light directing element attached to an instrument housing; and
at least one illumination device optically coupled to a peripheral portion of said at least one light directing element for providing illumination through said element to enable a user to view a target of interest.

2. An illumination system as recited in claim 1, wherein said light directing element includes a transparent window attached to said instrument housing.

3. An illumination system as recited in claim 2, wherein said peripheral portion of said transparent window is coated with a dispersive material to promote uniform illumination along said peripheral portion by said at least one illumination device.

4. An illumination system as recited in claim 1, wherein said medical diagnostic instrument is a sphygmomanometer.

5. An illumination system as recited in claim 1, wherein said light directing element is a speculum made from a light transmissive material, said at least one illumination device being coupled to a portion of said speculum.

6. An illumination system as recited in claim 1, including coupling means for optically coupling illumination from said at least one illumination device to said at least one light directing element.

7. An illumination system as recited in claim 6, wherein said coupling means includes at least one lens element disposed between said at least one illumination device and said at least one light directing element.

8. An illumination system as recited in claim 5, including coupling means for optically coupling illumination from said at least one illumination device to said at least one light directing element.

9. An illumination system as recited in claim 8, wherein said coupling means includes at least one lens element disposed between said at least one illumination device and said at least one light directing element.

10. An illumination system as recited in claim 5, wherein said medical diagnostic instrument is an otoscope.

11. An illumination system as recited in claim 1, wherein said light directing element is a mirrored surface, said illumination device being coupled to a portion of said mirrored surface.

12. An illumination system as recited in claim 11, wherein said medical diagnostic instrument is an intraoral dental mirror.

13. An illumination system as recited in claim 1, wherein said at least one illumination device is an LED.

14. An illumination system as recited in claim 13, wherein said LED is a white LED.

15. A medical diagnostic instrument comprising:
a housing;
a light directing element disposed in relation to said housing; and
at least one illumination device optically coupled to a peripheral portion of said at least one light directing element for providing illumination at a target of interest.

16. A medical diagnostic instrument as recited in claim 15, said instrument further including:
at least one pressure responsive element having a movable surface;
a movement mechanism disposed within said housing, said movement mechanism being disposed in relation to the movable surface of said pressure responsive element;
at least one indicating member which is caused to move from a first position to at least a second position when movement of said movable surface occurs; and
a dial face having indicia against which said indicating member moves.

17. A medical diagnostic instrument as recited in claim 16, wherein said light directing element includes a transparent window covering said dial face, said at least one illumination device being disposed in relation to a peripheral portion of said transparent window.

18. A medical diagnostic instrument as recited in claim 17, wherein said peripheral portion of said window is coated with a dispersive material for providing uniform illumination to said peripheral portion of said window.

19. A medical diagnostic instrument as recited in claim 15, including an inflatable sleeve which is wrappable about the limb of a patient, said sleeve being fluidly interconnected to said housing.

20. A medical diagnostic instrument as recited in claim 19, wherein said instrument is a syphygmomanometer.

21. A medical diagnostic instrument as recited in claim 15, wherein said instrument is an otoscope, said light directing element being a conically shaped speculum attachable to said housing, said at least one illumination device being coupled to a portion of said speculum to direct light from one end of said speculum to an opposite end thereof.

22. A medical diagnostic instrument as recited in claim 15, including coupling means for optically coupling illumination from said at least one illumination device to said light directing element.

23. A medical diagnostic instrument as recited in claim 22, wherein said coupling means includes at least one lens element disposed between said at least one illumination device and said light directing element.

24. A medical diagnostic instrument as recited in claim 21, including coupling means for optically coupling illumination from said at least one illumination device to said light directing element.

25. A medical diagnostic instrument as recited in claim 24, wherein said coupling means includes at least one lens element disposed between said at least one illumination device and said light directing element.

26. A medical diagnostic instrument as recited in claim 15, wherein said light directing element includes a mirror, said at least one illumination device being coupled to a peripheral portion of said mirror.

27. A medical diagnostic instrument as recited in claim 15, wherein said at least one illumination device is an illumination device, such as an LED.

28. A medical diagnostic instrument as recited in claim 27, wherein said LED is a white LED.

29. A pressure measuring device comprising:
a housing;
a pressure responsive member disposed within said housing, said pressure responsive member having a movable surface;
an indicating member which is caused to move based upon a change in said movable surface of said pressure responsive member;
a dial face having indicia against which said indicating member moves; and
an illumination system including at least one illumination device and at least one light directing element optically coupled to said illumination device for providing illumination to enable a user to view the dial face and indicating member during a measurement without requiring any ambient light.

30. A pressure measuring device as recited in claim 29, wherein said light directing element includes a transparent window covering said dial face.

31. A pressure measuring device as recited in claim 30, wherein said at least one illumination device is adjacently disposed to a portion of a peripheral edge of said window.

32. A pressure measuring device as recited in claim 31, wherein at least a portion of said peripheral edge is coated with a dispersive material.

33. A pressure measuring device as recited in claim 29, including activation means for automatically activating said at least one illumination device when said the movement of said movable surface of said pressure responsive member exceeds a predetermined amount.

34. A pressure measuring device as recited in claim 29, including timing means for automatically deactivating said at least one illumination device after a predetermined time interval.

35. A pressure measuring device as recited in claim 29, wherein said device is used for measuring blood pressure.

36. A pressure measuring device as recited in claim 35, wherein said device is releasably attachable to an inflatable sleeve.

37. A pressure measuring device as recited in claim 35, wherein said housing is mounted to one of a wall and a stand.

38. A pressure measuring device as recited in claim 34, wherein said deactivating means includes a timer circuit which is activated when said at least one illumination device is activated.

39. A pressure measuring device as recited in claim 29, including a protective bumper releasably attachable and covering the periphery of said housing, said at least one illumination device being disposed within said bumper.

40. A pressure measuring device as recited in claim 39, including powering means disposed within said releasably attachable bumper for powering said at least one illumination device.

41. A pressure measuring device as recited in claim 39, wherein a portion of said housing includes a notch sized for receiving said at least one illumination device.

42. A pressure measuring device as recited in claim 41, wherein at least a portion of said peripheral section of said window is coated with a dispersive material with the exception of a circumferential section of said peripheral portion adjacent said notch.

43. A pressure measuring device as recited in claim 29, wherein at least a part of said at least one light directing element is coated with a dispersive material.

44. A pressure measuring device as recited in claim 29, wherein said at least one illumination device is an LED.

45. A pressure measuring device as recited in claim 44, wherein said LED is a white LED.

46. A method for uniformly illuminating a medical target, said method comprising the steps of:
optically coupling at least one illumination device to a peripheral portion of a light directing element of a medical diagnostic instrument; and
illuminating said at least one illumination device so as to uniformly direct the light from said at least one illumination device towards said medical target.

47. A method as recited in claim 46, including the step of providing at least one lens element between said at least one illumination device and said light directing element.

48. A method as recited in claim 46, wherein said at least one illumination device is an LED.
